(19) United States
(12) Patent Application Publication
(10) Pub. No.: US 2011/0087084 A1
(43) Pub. Date: Apr. 14, 2011

(54) FACE MASK TYPE VITAL SIGNS MEASURING APPARATUS AND VITAL SIGNS MANAGEMENT SYSTEM USING THE SAME

(75) Inventors: Ji Wook JEONG, Daejeon (KR); Seung Hwan Kim, Daejeon (KR)

(73) Assignee: Electronics and Telecommunications Research Institute, Daejeon (KR)

(21) Appl. No.: 12/899,443

(22) Filed: Oct. 6, 2010

(30) Foreign Application Priority Data
Oct. 9, 2009 (KR) 10-2009-0096455
Feb. 12, 2010 (KR) 10-2010-0013341

(51) Int. Cl. A61B 5/145; A61B 5/00; A61B 5/097; A61B 5/01

(52) U.S. Cl. 600/364; 600/300; 600/529; 600/532; 600/549

(57) ABSTRACT

There are provided a face mask type vital signs measuring apparatus and a vital signs management system using the same. The face mask type vital signs measuring apparatus according to an exemplary embodiment of the present invention may include: a detector detecting a vital sign; an analyzer analyzing the detected vital sign; a display displaying the analyzed vital sign; a transmitter transmitting the analyzed vital sign; and a face mask with the detector, the analyzer, the display, and the transmitter attached thereto.

### CROSS-REFERENCE TO RELATED APPLICATIONS

This application claims the priorities of Korean Patent Application Nos. 10-2009-0096455 filed on Oct. 9, 2009 and 10-2010-0013341 filed on Feb. 12, 2010, in the Korean Intellectual Property Office, the disclosures of which are incorporated herein by reference.

### BACKGROUND OF THE INVENTION

1. Field of the Invention

The present invention relates to a face mask type vital signs measuring apparatus and a vital signs management system, and more particularly, to a face mask type vital signs measuring apparatus and a vital sign management system capable of managing a health index by measuring a vital sign in real time.

2. Description of the Related Art

In general, a demand for an auxiliary measuring apparatus freely and conveniently measuring and monitoring a vital sign in our daily lives or for exercise management is increasing on a daily basis.

With an aging tendency among the population of our society, concerns for the health management of the elderly are increasing. In particular, solitary elderly persons cannot easily operate simple home medical devices or measurement apparatuses, while the influence of diseases with which they must cope is important. Therefore, solitary elderly persons have recently become the main interest in terms of social health.

Further, research and development to simply and accurately measure a vital sign and analyze health indices such as respiration, pulse, etc. in real time in our daily lives is very much anticipated in terms of health management, particularly, obesity management for ordinary persons.

Meanwhile, a vital signs measuring method using a conductive fiber can be easily combined with articles such as clothes, etc. and has comparatively excellent electrical characteristics. Therefore, research and development of measurement of vital signs and the electrical signals thereof is in active progress.

Moreover, existing sensors such as an oxygen saturation sensor, a respiration sensor, a blood alcohol concentration sensor, an acceleration sensor, and a temperature sensor can be effectively utilized to measure an oxygen saturation signal, a respiration signal, a blood alcohol concentration signal, an acceleration signal, and a body temperature as the vital signs.

Accordingly, the current trend is for appearance of vital signs measuring apparatuses implemented by combining the articles such the clothes, etc. with various sensors.

Meanwhile, due to the steady technological development of a face mask generally used to prevent a circulatory disease or to prevent infection of the circulatory disease, the wearability of the face mask continues its trend of improvement so that the ordinary people have no aversion to wearing such a face mask.

Therefore, the necessity of such an apparatus, which can conveniently achieve a predetermined purpose such as emergency situation management and exercise management by using the face mask type settled as a general season-change health auxiliary device, is increasing.

That is, the development of a technology which is capable of demonstrating the characteristics of wireless communication, etc. to minimize restriction of a daily activity area by applying various sensors to the existing face mask type in consideration of the distinct characteristics of diagnosis and treatment environments is required.

### SUMMARY OF THE INVENTION

An aspect of the present invention provides a face mask type vital signs measuring apparatus capable of managing a health index by measuring a vital sign in real time.

An aspect of the present invention also provides a vital signs management system using a face mask type vital signs measuring apparatus capable of managing a health index by measuring a vital sign in real time.

According to an aspect of the present invention, there is provided a face mask type vital signs measuring apparatus including: a detector detecting a vital sign; an analyzer analyzing the detected vital sign; a display displaying the analyzed vital sign; a transmitter transmitting the analyzed vital sign; and a face mask with the detector, the analyzer, the display, and the transmitter attached thereto.

Herein, the vital sign may include at least one of oxygen saturation, respiration, blood alcohol concentration, acceleration, and body temperature.

Herein, the detector may include at least one of an oxygen saturation sensor, a respiration sensor, a blood alcohol concentration sensor, an acceleration sensor, and a temperature sensor.

Herein, the detector may detect the vital sign in a predetermined cycle.

Herein, the predetermined cycle of the detector may be adjusted in real time.

Herein, the analyzer may determine whether or not the detected vital sign is beyond a predetermined reference range.

Herein, the predetermined reference range of the analyzer may be adjusted in real time.

Herein, the display may display the detected vital sign by using at least one of a visual signal and an audio signal.

Herein, the display may display an alarm signal when the detected vital sign is beyond the predetermined reference range.

Herein, the alarm signal provided by the display may include at least one of a color, blinking light, and alarm sound.

Herein, the transmitter may transmit the detected vital sign to a server for vital signs management.

Herein, the transmitter may transmit the vital sign by using at least one of wired communication and wireless communication.

Herein, the transmitter may transmit the alarm signal to the server when the detected vital sign is beyond the predetermined reference range.

Herein, the face mask may include a resistive-conductive fiber.

Herein, the resistive-conductive fiber may include at least one of a conductive fiber, an elastic fiber, a carbon fiber, and a metal wire.

According to another aspect of the present invention, there is provided a vital signs management system using a face mask type vital signs measuring apparatus including: a face mask type vital signs measuring apparatus including a detector detecting a vital sign, an analyzer analyzing the detected vital sign, a display displaying the analyzed vital sign, a transmitter transmitting the analyzed vital sign, and a face mask with the detector, the analyzer, the display, and the transmitter attached thereto; and a vital signs management server receiving and managing the vital sign from the face mask type vital signs measuring apparatus.

Herein, the detector of the face mask type vital signs measuring apparatus may include at least one of an oxygen saturation sensor, a respiration sensor, a blood alcohol concentration sensor, an acceleration sensor, and a temperature sensor.

Herein, the analyzer of the face mask type vital signs measuring apparatus may determine whether or not the detected vital sign is beyond a predetermined reference range.

Herein, the display of the face mask type vital signs measuring apparatus may display the detected vital sign by using at least one of a visual signal and an audio signal.

Herein, the display of the face mask type vital signs measuring apparatus may display an alarm signal when the detected vital sign is beyond the predetermined reference range.

Herein, the transmitter of the face mask type vital signs measuring apparatus may transmit the alarm signal to the vital signs management server when the detected vital sign is beyond the predetermined reference range.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a block diagram of a face mask type vital signs measuring apparatus according to an exemplary embodiment of the present invention;

FIG. 2 is an exemplary diagram for describing a face mask type vital signs measuring apparatus according to an exemplary embodiment of the present invention;

FIG. 3 is an exemplary diagram for describing a respiration sensor in a face mask type vital signs measuring apparatus according to an exemplary embodiment of the present invention;

FIG. 4 is a measurement graph of a respiration sensor for describing the respiration sensor in a face mask type vital signs measuring apparatus according to an exemplary embodiment of the present invention; and

FIG. 5 is an exemplary diagram for describing a vital signs management system using a face mask type vital signs measuring apparatus according to an exemplary embodiment of the present invention.

### DETAILED DESCRIPTION OF THE PREFERRED EMBODIMENT

Since the present invention can be variously modified and have several embodiments, the exemplary embodiments are illustrated in the accompanying drawings and will be described in detail in the detailed description.

However, the present invention is not limited to the specific embodiments and should be construed as including all the changes, equivalents, and substitutions included in the spirit and scope of the present invention.

Terms such as ‘first’, ‘second’, etc. can be used to describe various components, but the components are not limited to the terms. Terms described in the specification is used to discriminate one component from other components. For example, the first component may be called the second component without departing from the scope of the present invention. Likewise, the second component may be called the first component. The term ‘and/or’ includes a combination of a plurality of items or any one of a plurality of terms.

Stated that any components are “connected” or “coupled” to other components, it is to be understood that the components may be directly connected or coupled to other components, but another component may intervene therebetween. On the other hand, stated that any components are “directly connected” or “directly coupled” to other components, it is to be understood that there is no another component therebetween.

The terms used in the specification is used to describe only specific embodiments and is not intended to limit the present invention. The singular forms are intended to include the plural forms unless the context clearly indicates otherwise. It will be further understood that the terms “comprises” or “have” used in this specification, specify the presence of stated features, steps, operations, components, parts, or a combination thereof, but do not preclude the presence or addition of one or more other features, numerals, steps, operations, components, parts, or a combination thereof.

Unless indicated otherwise, it is to be understood that all the terms used in the specification including technical and scientific terms has the same meaning as those that are understood by those who skilled in the art. It must be understood that the terms defined by the dictionary are identical with the meaning of the context of the related art, and they should not be ideally or excessively formally defined unless the context clearly dictate otherwise.

Exemplary embodiments of the present invention will now be described in detail with reference to the accompanying drawings. In describing the present invention, for ease of overall understanding, like elements refer to like reference numerals and like elements will not be described again.

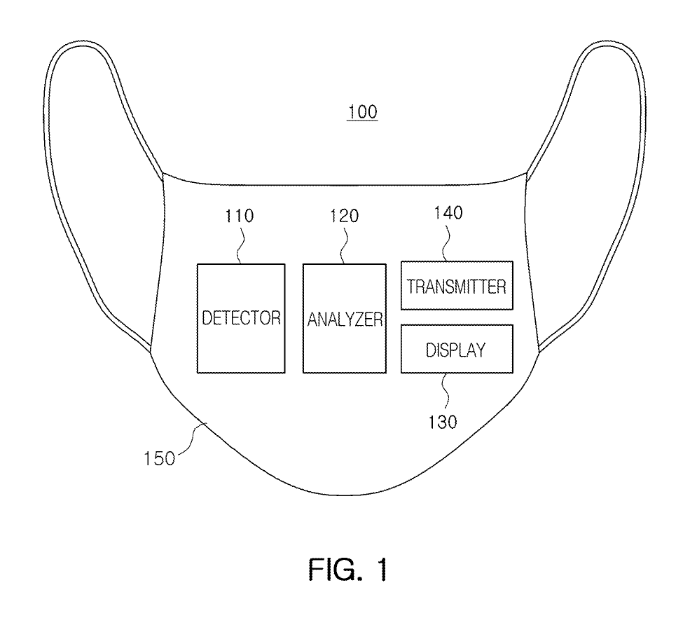

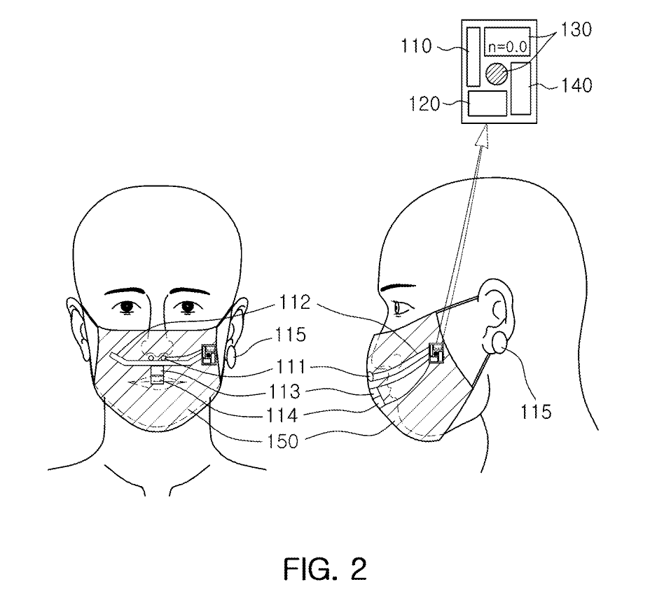

FIG. 1 is a block diagram of a face mask type vital signs measuring apparatus according to an exemplary embodiment of the present invention. FIG. 2 is an exemplary diagram for describing a face mask type vital signs measuring apparatus according to an exemplary embodiment of the present invention.

Alternately referring to FIGS. 1 and 2, the face mask type vital signs measuring apparatus 100 according to the exemplary embodiment of the present invention may include a detector 110 detecting a vital sign, an analyzer 120 analyzing the detected vital sign, a display 130 displaying the analyzed vital sign, a transmitter 140 transmitting the analyzed vital sign, and a face mask 150 attached thereto.

First, the vital sign may include at least one of oxygen saturation, respiration, blood alcohol concentration, acceleration, and body temperature. Various vital signs which can be measured by the sensors attached to the mask among phenomena generated in a human body may be included in the vital sign according to the exemplary embodiment of the present invention.

That is, oxygen saturation, respiration, blood alcohol concentration, acceleration, and body temperature are exemplified as the vital sign for description and it will be apparent to those skilled in the art that the vital sign is not limited to the vital signs which can be measured in the present invention.

Next, the detector 110 may include at least one of an oxygen saturation sensor 115, a respiration sensor 112, a blood alcohol concentration sensor 113, an acceleration sensor 114, and a temperature sensor 111. As such, the sensor measuring the vital sign may include various sensors.

In particular, a method for measuring the blood alcohol concentration includes a method using the respiration and a method using blood. Of the two methods, in the case that the method using respiration is applied, the blood alcohol concentration will be one of the most characteristic vital signs which can be measured by the face mask type vital signs measuring apparatus.

The detector 110 may detect the vital sign in a predetermined cycle and the predetermined cycle of the detector 110 may be adjusted in real time.

For, example, the vital sign may be once measured every minute in the predetermined cycle and the vital sign may also be measured once every 30 seconds by adjusting the predetermined cycle in real time. Since the vital sign measurement cycle can be adjusted in real time, it will be able to cope with various situations.

Next, the analyzer 120 may determine whether or not the detected vital sign is beyond a predetermined reference range and the predetermined reference range of the analyzer may be adjusted in real time.

In general, since a normal range for the vital sign can be predicted, a normal range for each vital sign will be able to be determined as the predetermined reference range. That is, the analyzer will be able to easily determine whether the detected vital sign is within the predetermined range (normal range).

For example, when a respiration rate per minute of 60 to 80 times is set as the normal range, the predetermined reference range of the respiration rate may be determined as being 60 to 80 times per minute. Therefore, by comparing the predetermined reference range with a measurement result of respiration rate for one minute using the respiration sensor, it may be determined as to whether or not the respiration rate is within the normal range.

Further, according to present regulations in some jurisdictions, when the blood alcohol concentration of a driver is 0.03% or more, the driver is subjected to penalty under the law. Therefore, in this case, the predetermined reference range may be set to be 0.00 to 0.03%. On the contrary, when the blood alcohol concentration measured by the blood alcohol concentration sensor is higher than 0.03%, it is determined that the blood alcohol concentration is beyond the normal range. Therefore, in this case, an indication corresponding to the determination may be displayed.

Moreover, since the predetermined reference range may be adjusted in real time, the predetermined reference range may be changed and applied in real time. Since the reference range may be differently applied depending on various cases including cases in which the human body is in a normal state, in lying, and during exercise and the reference range is applicable in real time, immediate action will be available depending on the state of the human body.

Next, the display 130 may display the detected vital sign by using at least one of a visual signal and an audio signal. Accordingly, a user will be able to take a rest, continue the exercise, or perform other actions while observing the displayed measurement value of the vital sign. In other words, the user will be able to make various determinations of actions depending on the state of the human body.

For example, the display 130 may display the measurement values detected by the oxygen saturation sensor 115, the respiration sensor 112, the blood alcohol concentration sensor 113, the acceleration sensor 114, and the temperature sensor 111 through a screen which is the visual signal and a language which is the audio signal, which are recognizable by the user.

Meanwhile, the display 130 may display an alarm signal when the detected vital sign is beyond the predetermined reference range and the alarm signal provided by the display 130 may include at least one of a color, blinking light, and alarm sound.

For example, in the case that the value measured by the alcohol concentration sensor is beyond the predetermined reference range when the measurement value is higher than 0.03%, the fact that the measurement value is beyond the predetermined reference range may be notified to the user through display of different colors or blinking lights as a visual alarm signal or the user may be alerted by generating the alarm sound as an audible alarm signal.

Next, the transmitter 140 may transmit the detected vital sign to a server for vital signs management. Further, the transmitter 140 may transmit the vital sign by using at least one of wired communication and wireless communication.

That is, the measurement values detected by the oxygen saturation sensor 115, the respiration sensor 112, the blood alcohol concentration sensor 113, the acceleration sensor 114, and the temperature sensor 111 may be recognized by the user and transmitted to the vital signs management server installed in a medical institution or a public institution.

For example, when the value measured by the oxygen saturation sensor 115 is cyclically transmitted to the vital signs management server installed in the medical institution, the vital signs management server analyzes and stores the measurement value so as to perform centralized, systematic personal health management.

Moreover, the transmitter 140 may transmit data to the vital signs management server through wireless communication or wired communication. That is, the data may be transmitted using at least one of Bluetooth®, WLAN, WiMAX, WCDMA, and LTE and transmitted by wired communication using a copper wire and an optical fiber.

Further, the transmitter 140 may transmit the alarm signal to the server when the detected vital sign is beyond the predetermined reference range. For example, when the value measured by the oxygen saturation sensor is beyond the predetermined reference range, the transmitter transmits the alarm signal to the vital signs management server so as to take rapid action such as the allocation of an ambulance according to an emergency medical service system of the medical institution.

Next, the face mask 150 attached with the detector 110, the analyzer 120, the display 130, and the transmitter 140 may include a resistive-conductive fiber. The resistive-conductive fiber may include at least one of a conductive fiber, an elastic fiber, a carbon fiber, and a metal wire.

Although the resistive-conductive fiber of the face mask 150 is included in the face mask because it is a fiber type, the user is not inconvenienced by its inclusion therein and may thus conveniently use the face mask type vital signs measuring apparatus.

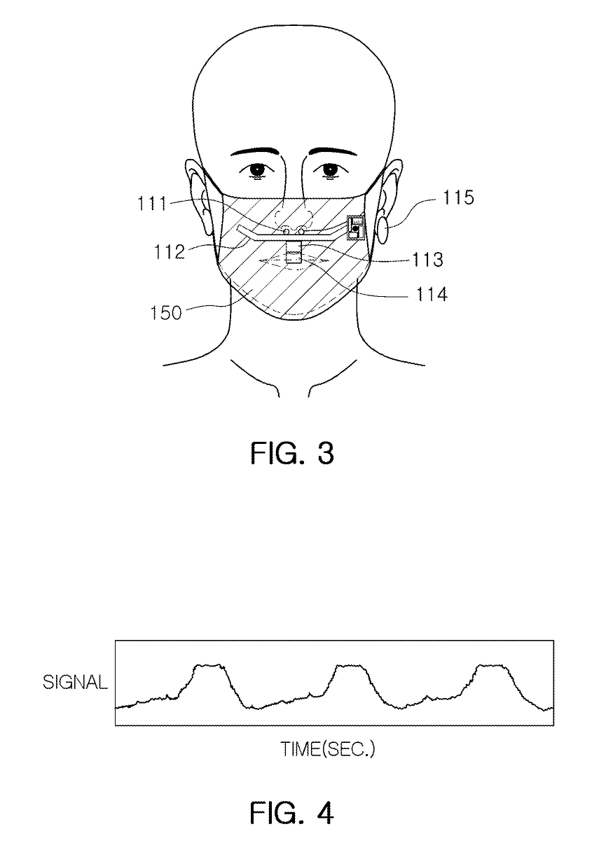

FIG. 3 is an exemplary diagram for describing a respiration sensor in a face mask type vital signs measuring apparatus according to an exemplary embodiment of the present invention. FIG. 4 is a measurement graph of a respiration sensor for describing the respiration sensor in a face mask type vital signs measuring apparatus according to an exemplary embodiment of the present invention.

Alternately referring to FIGS. 3 and 4, in the face mask type vital signs measuring apparatus 100 according to the exemplary embodiment of the present invention, the respiration sensor 112 is included in the detector 110 and may be mounted at a portion of the face mask where the respiration sensor 112 can detect air introduced or discharged through the nose.

Accordingly, the number of times of expiration and inspiration occurred by the respiration of the human body may be detected and thus, the detected number of expirations and inspirations may be analyzed as a measurement value once every 15, one minute or once every 30 seconds.

The respiration sensor for the measurement may adopt various kinds of sensors, but for ease of description, a respiration sensor having a characteristic in which the length thereof is increased in the case of expiration and decreased in the case of inspiration by detecting the expiration and inspiration phases of the respiration will be described as an example.

Referring to FIG. 4, by using a sensor in which a resistance value (signal) thereof is decreased when the length of the respiration sensor is decreased in the case of the inspiration and the resistance value of the respiration sensor is increased when the length of the respiration sensor is increased in the case of the expiration, a resistance value measured depending on time may be acquired.

As shown in the graph, resistance is cyclically increased as time elapses and an increase in resistance indicates the generation of the expiration. Accordingly, a respiration rate can be acquired by measuring the number of peaks of the graph on the basis of a predetermined time.

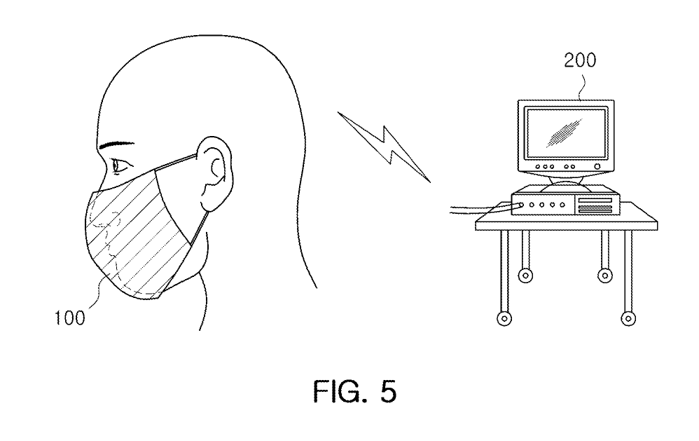

FIG. 5 is an exemplary diagram for describing a vital signs management system using a face mask type vital signs measuring apparatus according to an exemplary embodiment of the present invention.

Referring to FIG. 5, the vital signs management system using the face mask type vital signs measuring apparatus according to the exemplary embodiment of the present invention may include a face mask type vital signs measuring apparatus 100 including a detector detecting a vital sign, an analyzer analyzing the detected vital sign, a display displaying the analyzed vital sign, and a transmitter transmitting the analyzed vital sign, and a vital signs management server 200 receiving and managing the vital sign from the face mask type vital signs measuring apparatus.

The detector of the face mask type vital signs measuring apparatus may include at least one of an oxygen saturation sensor, a respiration sensor, a blood alcohol concentration sensor, an acceleration sensor, and a temperature sensor.

The analyzer of the face mask type vital signs measuring apparatus may determine whether or not the detected vital sign is beyond a predetermined reference range.

The display of the face mask type vital signs measuring apparatus may display the detected vital sign by using at least one of a visual signal and an audio signal.

Further, the display of the face mask type vital signs measuring apparatus may display an alarm signal when the detected vital sign is beyond the predetermined reference range.

The transmitter of the face mask type vital signs measuring apparatus may transmit the alarm signal to the vital signs management server 200 when the detected vital sign is beyond the predetermined reference range.

As set forth above, according to exemplary embodiments of the present invention, a face mask type vital signs measuring apparatus and a vital signs management system using the same can manage a health index by measuring a vital sign in real time and since they use a face mask capable of minimizing inconvenience in our daily life, they can be conveniently used.

In particular, the face mask type vital signs measuring apparatus can analyze, in real time, vital signs such as oxygen saturation, respiration, blood alcohol concentration, acceleration, and body temperature measured by sensors and immediately display the vital signs in the case of an emergency situation so as to allow action to be taken to remedy the emergency situation. Further, since the face mask type vital signs measuring apparatus can transmit the vital signs to an additional vital signs management server, it can take systematic action against the emergency.

While the present invention has been shown and described in connection with the exemplary embodiments, it will be apparent to those skilled in the art that modifications and variations can be made without departing from the spirit and scope of the invention as defined by the appended claims.

What is claimed is:

1. A face mask type vital signs measuring apparatus, comprising:
a detector detecting a vital sign;
an analyzer analyzing the detected vital sign;
a display displaying the analyzed vital sign;
a transmitter transmitting the analyzed vital sign; and
a face mask with the detector, the analyzer, the display, and the transmitter attached thereto.

2. The face mask type vital signs measuring apparatus of claim 1, wherein the detector includes at least one of an oxygen saturation sensor, a respiration sensor, a blood alcohol concentration sensor, an acceleration sensor, and a temperature sensor.

3. The face mask type vital signs measuring apparatus of claim 1, wherein the detector detects the vital sign in a predetermined cycle.

4. The face mask type vital signs measuring apparatus of claim 3, wherein the predetermined cycle of the detector is adjustable in real time.

5. The face mask type vital signs measuring apparatus of claim 1, wherein the analyzer determines whether or not the detected vital sign is beyond a predetermined reference range.

6. The face mask type vital signs measuring apparatus of claim 5, wherein the predetermined reference range of the analyzer is adjustable in real time.

7. The face mask type vital signs measuring apparatus of claim 1, wherein the display displays the detected vital sign by using at least one of a visual signal and an audio signal.

8. The face mask type vital signs measuring apparatus of claim 5, wherein the display displays an alarm signal when the detected vital sign is beyond the predetermined reference range.

9. The face mask type vital signs measuring apparatus of claim 8, wherein the alarm signal provided by the display includes at least one of a color, blinking light, and alarm sound.

10. The face mask type vital signs measuring apparatus of claim 1, wherein the transmitter transmits the detected vital sign to a server for vital signs management.

11. The face mask type vital signs measuring apparatus of claim 1, wherein the transmitter transmits the vital sign by using at least one of wired communication and wireless communication.

12. The face mask type vital signs measuring apparatus of claim 5, wherein the transmitter transmits the alarm signal to the server when the detected vital sign is beyond the predetermined reference range.

13. The face mask type vital signs measuring apparatus of claim 1, wherein the face mask includes a resistive-conductive fiber.

14. The face mask type vital signs measuring apparatus of claim 13, wherein the resistive-conductive fiber includes at least one of a conductive fiber, an elastic fiber, a carbon fiber, and a metal wire.

15. A vital signs management system, comprising:
a face mask type vital signs measuring apparatus including a detector detecting a vital sign, an analyzer analyzing the detected vital sign, a display displaying the analyzed vital sign, a transmitter transmitting the analyzed vital sign, and a face mask with the detector, the analyzer, the display, and the transmitter attached thereto; and
a vital signs management server receiving and managing the vital sign from the face mask type vital signs measuring apparatus.

16. The vital signs management system of claim 15, wherein the detector of the face mask type vital signs measuring apparatus includes at least one of an oxygen saturation sensor, a respiration sensor, a blood alcohol concentration sensor, an acceleration sensor, and a temperature sensor

17. The vital signs management system of claim 15, wherein the analyzer of the face mask type vital signs measuring apparatus determines whether or not the detected vital sign is beyond a predetermined reference range.

18. The vital signs management system of claim 15, wherein the display of the face mask type vital signs measuring apparatus displays the detected vital sign by using at least one of a visual signal and an audio signal.

19. The vital signs management system of claim 17, wherein the display of the face mask type vital signs measuring apparatus displays an alarm signal when the detected vital sign is beyond the predetermined reference range.

20. The vital signs management system of claim 17, wherein the transmitter of the face mask type vital signs measuring apparatus transmits the alarm signal to the vital signs management server when the detected vital sign is beyond the predetermined reference range.
